(19) United States
(12) Patent Application Publication
(10) Pub. No.: US 2020/0282229 A1
(43) Pub. Date: Sep. 10, 2020

(54) Wearable Light-Emitting Diode Therapy Device

(71) Applicant: Illumiheal Inc., Shoreline, WA (US)

(72) Inventor: Kristine Marzolf, Shoreline, WA (US)

(21) Appl. No.: 16/813,209

(22) Filed: Mar. 9, 2020

Related U.S. Application Data
(60) Provisional application No. 62/816,157, filed on Mar. 10, 2019.

(51) Int. Cl. A61N 5/06; G16H 40/67

(52) U.S. Cl. CPC A61N 5/0616; G16H 40/67; A61N 2005/0644; A61N 2005/0645; A61N 2005/0632; A61N 2005/0651

(57) ABSTRACT

A mitten for providing light therapy comprising a plurality of a light-emitting diodes (LEDs) disposed on a bottom surface of the mitten and a digital display and one or more input switches disposed on a top surface of the mitten. The mitten further includes a wireless transmitter, a power source, a processor, and a memory disposed on the top surface of the mitten, the processor being configured to set a photobiomodulation treatment mode for the plurality of light-emitting diodes and transmit usage data through the wireless transmitter.

This application claims priority from U.S. Provisional Application No. 62/816,157 filed on 10 Mar. 2019, which is herein incorporated by reference in its entirety for all purposes.

### BACKGROUND

### Technical Field

Embodiments generally relate to animal therapy equipment. More specifically, a wearable, light-emitting diode smart mitten worn by a user, which provides pain relief of musculoskeletal, connective tissue and skin trauma on an animal.

### Background

The pet industry has seen amazing growth over the past years. Consumer-facing, natural pet products are a booming industry, with middle-class owners spending to give their animals better quality of life, from holistic food and natural flea and tick repellents to toys made from non-synthetic fibers. Also on the rise are pet insurance, advanced pet surgical procedures, digital devices, and specialty pet services.

Pets are living longer than ever, but as they age, they become prone to ailments and chronic illness. Arthritis, skin infections, and musculoskeletal disorders are among the top medical conditions for dogs and cats. As conditions worsen, treatment options are limited to restrictive exercise, surgical procedures, and medications that often lead to other health complications and an overall lack of vitality. To treat these conditions, pet owners face the inconvenience of many costly veterinary visits, which are both time-consuming for them and disruptive for their pet—often leaving the pet nervous and stressed.

### BRIEF DESCRIPTION OF THE DRAWINGS

The accompanying drawings are incorporated herein and form a part of the specification.

FIG. 1 illustrates a top and bottom view of a light-therapy mitten device, according to an example embodiment.

FIG. 2 illustrates a top view of a light-therapy mitten device worn on a user's hand, according to an example embodiment.

FIG. 3 illustrates a bottom view of a light-therapy mitten device worn on a user's hand, according to an example embodiment.

FIG. 4 illustrates a light-therapy mitten device being used on a pet, according to an example embodiment.

### DETAILED DESCRIPTION

Provided herein are embodiments of wearable light-emitting diode mitten for providing pain relief of musculoskeletal, connective tissue, and skin trauma on an animal.

Low Level Light Therapy (LLLT) utilizes penetrating photonic energy to achieve psychological and bio chemical changes within the targeted tissues. Also known as “photobiomodulation therapy” (PBMT), this is a form of light therapy that results in beneficial therapeutic outcomes including but not limited to the alleviation of pain or inflammation, immunomodulation, and promotion of wound healing and tissue regeneration. Photobiomodulation therapy (PBMT) is the therapeutic use of light, absorbed by the chromosphere found in the body to stimulate non-harmful and non-thermal reactions within the cell that result in a beneficial therapeutic outcome.

For use in pets, current devices are limited to hand held units such as wands, pins, or and thick pads. These instruments do not provide the best delivery of treatment on a moving pet. It is ideal that a pet remains comfortable and still while receiving treatment, otherwise its easy to treat the wrong location. Current devices are also limited by few treatment modes, wavelengths, data insight, power efficiency and portability.

The embodiments described herein are directed to a wearable low-level light therapy smart mitten device worn by a user on their hand. The mitten is applied directly to a treatment area on a pet to help alleviate pain and discomfort, decrease inflammation, speed wound healing, treat chronic skin conditions, and accelerate post-surgical rehabilitation time. In particular embodiments, the mitten device is put on a user's left or right hand, as it is ambidextrous, then turned on by selecting a treatment mode. The device uses light diodes located on the palm of the mitten, which emit medical grade red, infrared, and/or blue lights which are associated with faster healing/pain relief of animals suffering from musculoskeletal, connective tissue and skin trauma. The device may further wireless short-range communications technology to deliver data insights to a computer or other smart device, and may operate on a rechargeable battery. The device can also be attached directly to the treatment area on the pet with fasteners.

Embodiments provide for a device that can be used to administer comfortable, easy to use therapeutic treatment for pets. The therapy can be an alternative to invasive treatments and pain management without the use of surgery or drugs. The device is a smart wearable therapeutic pet care device that uses low-level light therapy (LLLT) and gentle heat to reduce pain, speed wound healing, decrease inflammation, treat chronic skin conditions, and accelerate post-surgical rehabilitation time. The mitten device material may be flexible and naturally form to the treatment area, and is rechargeable and portable making it easy to use anywhere.

Embodiments include is a wearable ambidextrous mitten that can be hand-worn then applied to or attached directly to a body part or treatment area. The hand-worn mitten allows for greater control over the animal when receiving treatment. The device consists of a minimum of four apertures on opposite sides, which allow for the use of hands-free application by use of fasteners. In particular embodiments, the device is comprised of four layers including an exterior cover, electronics, protective electronic sheathing, and interior lining.

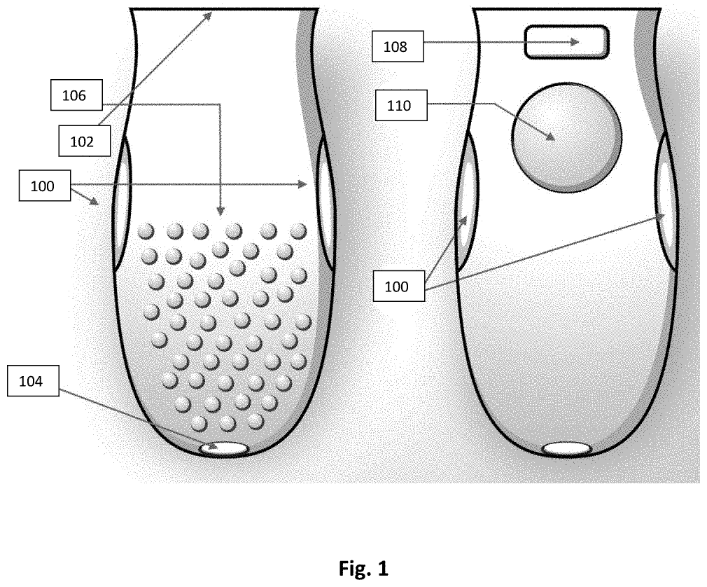

FIG. 1 is an illustrative representation showing the top and bottom view of a mitten device, in accordance with an embodiment of the present invention. The mitten is a wearable low-level light therapy device that uses light-emitting diodes for phototherapy with associated treatment methods to expose an animal's body part to various multi-wavelengths of lights and gentle heat, thus promoting healing and tissue regeneration, alleviating pain and discomfort, and decreasing inflammation in animals suffering from musculoskeletal, connective tissue and skin trauma.

The device is in a form of a mitten worn by a user. In particular embodiments, the device features two openings (100) on opposite sides that allow the user to slip their thumb through, allowing slight use of the hand. The two openings provide dexterity to the device allowing the user to choose the hand to put it on. The opening for the user to insert their hand (102) may be fastened by methods such as, but not limited to, a fastening belt, hook and loop fabric (e.g., Velcro®), an elastic band, etc. The front hole (104), allows the use of fasteners that can be attached directly to the animal without the user wearing the device.

The bottom of the device features light emitting diodes (106) which emit therapeutic infrared, blue and/or red light. The devices may use multiple wavelength LEDs matrices using various predefined treatment modes to administer light and heat to the treatment location. In particular embodiments, the light emittance of two or more light emitters is within a range which is enough to provide therapeutic effects through photobiomodulation. The top of the device may feature embedded electronics and power source (108). The electronics may include a computer processor and a memory for running embedded software for configuring the device and conducting wireless communications. The device may contain one or more power source(s) for efficient light distribution. Power sources may include, for example, one or more batteries, one or more power supplies connected to a power outlet, etc.

The top may also include a control panel (110) that may be in the form of a display. The user control panel may consist of multiple operational controls that signal the various predefined therapeutic treatment modes and device operation. Electronics component 108 may further include wireless short-range communications technology (e.g. Bluetooth, WiFi, RF, etc.) that transfers usage data from the device to computer or smart devices where data can be viewed and shared. For example, the device can communicate with a smartphone and update an animal's medical records based on data received. The material used for the device must be soft in nature and allows for a slight stretch over a user's hand. The number of light-emitting diodes may vary based on best application.

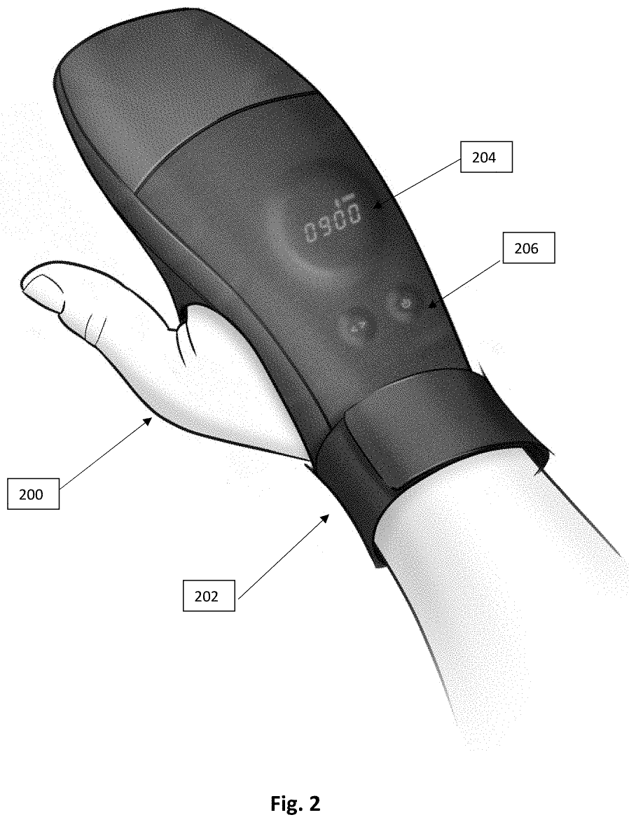

FIG. 2 is an illustrative representation showing the device worn on a user's hand seen from the top, in accordance with an embodiment of the present invention. This example is provided to allow the reader of this document understand the top features of the device and how it is worn by a user. The thumb (200) is seen sticking out of one of the holes featured on the mitten. In this example, the fastening method is a wrist strap (202) which wraps around a user's wrist when wearing the device. FIG. 2 further shows an example control panel screen (204) and control panel settings input buttons (206). The control panel display and inputs may be used to configure and/or select preconfigured photobiomodulation treatment modes of the LEDs. For example, the LED matrix may include LEDs that emit different wavelengths of light. The embedded electronics may be configured to activate any suitable subset of the LEDs in any suitable intensity, frequency, and duration. For example, the device may only emit one wavelength (e.g., blue), two wavelengths (red and blue), or all three wavelengths (blue, red, and infrared) in any sequence.

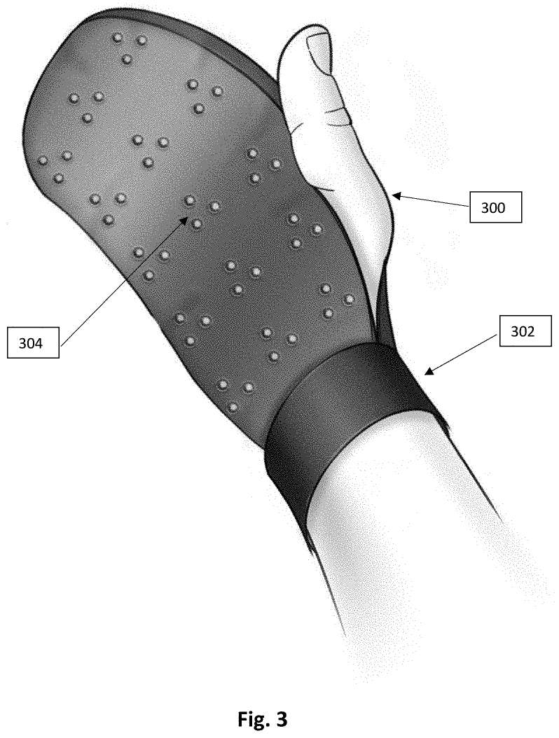

FIG. 3 is an illustrative representation showing an illustration for the device worn on a user's hand seen from the bottom, in accordance with an embodiment of the present invention. This example is provided to allow the reader of this document understand the bottom features of the device and how it is worn by a user. The thumb (300) is seen sticking out of one of the holes featured on the mitten. In this example, the fastening method is a wrist strap (302) which wraps around a user's wrist when wearing the device. The bottom features a soft surface with multiple light emitting diodes (304) built into it. The number of lights may vary and does not have to be specific to what is seen in the depiction. The set-up and arrangement of the lights may also vary.

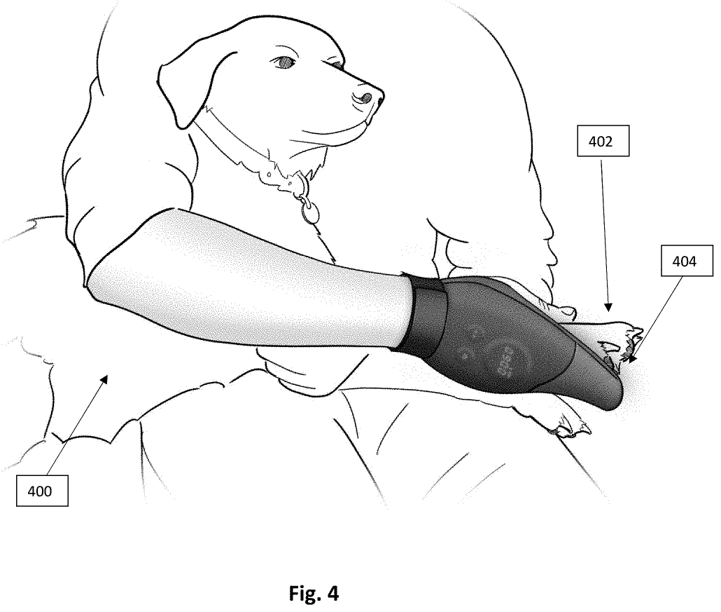

FIG. 4 is an illustrative representation showing an illustration of the device being used on a pet, in accordance with an embodiment of the present invention. This example is provided to allow the reader of this document understand the use of the device on a pet. The user is seen holding a dog (400) that has been injured. The mitten flexes with the hand and is seen here wrapping around the dog's leg (402). A red light (404) is seen emitting from around where the mitten touches the dog's leg. The glove may be used for other animals and other injured body parts not depicted in this figure. For example, if a dog injured their back, the user may place the glove on the dog to alleviate their backpain.

While the invention has been described herein with reference to exemplary embodiments for exemplary fields and applications, it should be understood that the invention is not limited thereto. Other embodiments and modifications thereto are possible, and are within the scope and spirit of the invention. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments may perform functional blocks, steps, operations, methods, etc. using orderings different than those described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

The breadth and scope of the invention should not be limited by any of the above-described exemplary embodiments, but should be defined only in accordance with the following claims and their equivalents.

What is claimed is:

1. A mitten for providing light therapy, the mitten comprising:
two openings on opposite sides of the mitten for inserting a thumb;
a plurality of a light-emitting diodes (LEDs) disposed on a bottom surface of the mitten;
a digital display and one or more input switches disposed on a top surface of the mitten;
a wireless transmitter;
a processor and a memory disposed on the top surface of the mitten, the processor being configured to set a photobiomodulation treatment mode for the plurality of light-emitting diodes and transmit usage data through the wireless transmitter; and
a power source disposed on the top surface of the mitten, the power source configured to provide electricity for the plurality of light-emitting diodes, the display, the processor and the memory, and the wireless transmitter.

2. The mitten of claim 1, wherein the LEDs are disposed in a matrix configuration, and wherein a portion of the LEDs provides one wavelength of light and another portion of the LEDs provides a different wavelength of light.

3. The mitten of claim 2, wherein LEDs are comprised of three types providing three different wavelengths of light, the three different wavelengths comprising infrared, blue, and red light.

4. The mitten of claim 1, wherein the setting a photobiomodulation treatment mode for the plurality of light-emitting diodes comprises configuring at least one of an intensity of the LEDs, a duration of treatment, and one or more wavelengths of lights to emit.

5. The mitten of claim 1, further comprising a fastener for securing the mitten to the user's hand.

6. The mitten of claim 5, wherein the fastener comprises at least one of a belt, a hook and loop fabric, and an elastic band.

7. The mitten of claim 1, further comprising an opening on a front side of the mitten for fastening the mitten directly to therapy recipient.

8. The mitten of claim 1, wherein the power source comprises at least one of a battery and a power supply plugged to a power outlet.

9. The mitten of claim 1, wherein the wireless transmitter is configured to transmit data to a smartphone.
